(19) United States
(12) Patent Application Publication
(10) Pub. No.: US 2002/0045256 A1
(43) Pub. Date: Apr. 18, 2002

(54) Method for enhancing primordial germ cell number

(75) Inventors: Guang-Quan Zhao, Plano, TX (US); Ying Ying, Providence, RI (US)

Correspondence Address: SENNIGER POWERS LEAVITT AND ROEDEL, ONE METROPOLITAN SQUARE, 16TH FLOOR, ST LOUIS, MO, 63102, US

(73) Assignee: The Curators Of the University of Missouri

(21) Appl. No.: 09/978,877

(22) Filed: Oct. 16, 2001

Related U.S. Application Data
(60) Provisional application No. 60/241,395, filed on Oct. 18, 2000.

(51) Int. Cl. C12N 5/08; C12N 5/06

(52) U.S. Cl. 435/325; 435/366

(57) ABSTRACT

Methods for enhancing the generation of primordial germ cells in pluripotent embryonic cells by contacting the pluripotent cells with at least one 60A class BMP protein and at least one DPP class BMP protein are provided. Also provided are methods for treating sterility and for the introduction of at least one transgene into the germ line of an animal.

### GOVERNMENT INTERESTS

This invention was made with government support under grant number 5 R29 HD36218 awarded by the National Institutes of Health. The government has certain rights in this invention.

### BACKGROUND

Prior to gastrulation, the mammalian embryo consists of three distinct cell lineages which are established during the peri-implantation stage of development, the epiblast (also known as the primitive ectoderm), the extraembryonic endoderm, and the trophectoderm. The epiblast, which will eventually develop into the entire fetus as well as the extraembryonic mesoderm and the amnion ectoderm, is a cup-shaped epithelium apposed on its open end to the extraembryonic ectoderm, a trophectoderm derivative. Both the epiblast and the extraembryonic ectoderm are covered by visceral endoderm, which is part of the extraembryonic endoderm lineage (Hogan et al., Manipulating the Mouse Embryo, Cold Spring Harbor Laboratory Press, 1994).

The primordial germ cells (PGCs) of mammals are derived from a part of the population of proximal epiblast cells. The earliest time that primordial germ cells can be identified in embryos is mid-gastrulation (Ginsburg et al., Development, 110;521-528, 1990). Prior to gastrulation, precursors of PGCs are located in the extreme proximal region of the epiblast adjacent to the extraembryonic ectoderm. At 7.25-7.5 day post coitus (dpc) in the mouse, PGCs are seen as a cluster of cells located in the extraembryonic region of the primitive streak. The PGCs subsequently migrate through the base of the allantois, the endoderm of the hindgut, and the mesenchyme of the mesentery to the genital ridge by 10.5 dpc. Before 11.5 dpc, there is little morphological difference between male and female gonads, although genes involved in sexual differentiation start to be expressed differentially (McLaren, Current Biol. 8:R175-R177, 1988; Trends Genet. 4:153-157, 1998)

A number of investigators have attempted to identify germ cells or germ cell determinants in early mammalian embryos without success because cells that are able to give rise to germ cells also serve as progenitors for other cell lineages (Kelly, J. Exp. Zool. 200:365-376, 1977; Gardner and Cockroft, Development 125:2397-2402, 1998). Therefore, a strict germ cell lineage has not been recognized in pre-implantation embryos.

Several research groups have generated data regarding the cellular mechanisms for cell fate determination of mammalian PGCs after implantation. Lawson and Hage (Ciba Found. Symp. 182:62-84, 1994) using lineage-tracing techniques found that only epiblast cells of the proximal region (i.e. close to the extraembryonic ectoderm) at 6.0-6.5 dpc in the mouse embryo contribute to PGCs. Moreover, the descendants of a single labeled cell in the proximal epiblast were found in germ cells and in several somatic lineages as well, so that no labeled cells contributed solely to the germ cell lineage. Therefore, during early gastrulation the cell fate of the PGCs is not completely fixed.

Based on data obtained by Lawson and Hage, supra, Tam and Zhou (Devel. Biol. 178:124-132, 1996) carried out a series of epiblast transplantation experiments in mouse embryos involving homotypical (proximal to proximal and distal to distal) and heterotypical (proximal to distal and distal to proximal) transplantation of epiblast cells. The authors found that before 6.5 dpc, cells of the distal epiblast which normally give rise to the neuroectoderm and surface ectodern, were able to generate PGCs if they were transplanted in close proximity with extraembryonic ectoderm. They found, however, that cells of the proximal epiblast never gave rise to PGCs if they were transplanted into the distal region far away from the extraembryonic ectoderm. Therefore, before 6.5 dpc, epiblast cells at different locations have the potency to generate PGCs only if they are close to the extraembryonic ectoderm, suggesting that signals from the extraembryonic ectoderm are critical for PGC fate specification.

The bone morphogenetic proteins (BMPs) are members of a large highly conserved family of extracellular polypeptide signaling molecules related to transforming growth factor-β. Greater than 20 members of BMPs have been identified in organisms ranging from sea urchins to mammals. The name bone morphogenetic proteins is due to the fact that the proteins were originally purified from a demineralized bovine bone preparation that induced ectopic cartilage and endochondral bone when implanted in experimental animals. There is now evidence, however, that these molecules regulate diverse biological processes including cell proliferation, apoptosis, differentiation, cell-fate determination, and morphogenesis. In particular, there is evidence that vertebrate BMPs are involved in the development of nearly all organs and tissues as well as in the establishment of the basic body plan during embryonic development.

BMP proteins are processed from a preprotein and dimerized to form the mature protein. Within the vertebrate BMP family there are several distinct classes. For example, BMP2 and BMP4 are most closely related to Drosophila decapentaplegic (DPP) and are herein collectively referred to as the DPP class of BMP proteins. BMP5, BMP6, BMP7, BMP8A and BMP8B are most closely related to glass bottom boat-60A in Drosophila and are collectively referred to herein as the 60A class BMP proteins. It is generally believed that the heterodimers of the 60A and DPP classes are the most potent BMPs for signal transduction and that the homodimers of these two groups of BMPs are functionally interchangeable for most if not all of their biological activities. Thus, it has been thought that combinations of 60A and DPP class proteins would have no advantage over the individual proteins alone. For example, Nishimatsu and Thomsen (Mech. Develop. 74:75-88, 1998) reported that while BMP2/7 heterodimers acted as mesoderm inducers, mixtures of BMP7 and either BMP2 or BMP4 homodimers did not.

Lawson et al. (Genes Develop. 13:424-436, 1999) reported that extraembryonic ectoderm-produced BMP4 was required for PGC generation in mice. Bmp4 expression in the extraembryonic ectoderm starts at 5.5 dpc and increases as the embryo develops. On several genetic backgrounds, all Bmp4 null (homozygous) mutants failed to generate PGCs. The authors concluded, therefore, that Bmp4 is absolutely required for PGC generation. The present inventors (Ying et al. Molec. Endocrinol. 14:1053-1063, 2000) reported that Bmp8b, which is also expressed in the extraembryonic ectoderm, is required for PGC generation. Thus, it is known that both Bmp4 and Bmp8b appear to needed for the generation of PGCs in vivo. The mechanism by which BMP4 and BMP8B act, however, is unknown. Until the present discovery it was unknown if either BMP4 or BMP8B was sufficient, by itself, to cause primordial germ generation; if both BMP4 and BMP8B were required; or if additional, unknown factors were also required. In addition, it was unknown if BMPs acted only as heterodimers, only as homodimers or as a combination of heterodimers and homodimers. Further, it was unknown whether BMP4 and BMP8B acted directly to cause the generation of primordial germ cells or acted through one or more intermediaries. The present inventors have surprisingly found that contacting pluripotent embryonic cells with a combination of a 60A class BMP protein and a DPP class BMP protein will result in a high percentage of those cells developing into primordial germ cells. The present invention, therefore, provides the first method for enhancing the generation of primordial germ cells from pluripotent embryonic cells.

### SUMMARY

Disclosed herein is a method for enhancing the generation of primordial germ cells from pluripotent embryonic cells by contacting the cells with a combination of at least one 60A class BMP protein and at least one DPP class BMP protein. The ability to influence the fate of pluripotent embryonic cells is of critical importance in the study of embryonic development and in particular the development of germ cells. In addition, the present method provides a means for treating sterility in vertebrate animals due to a deficiency in one or more BMP proteins. The methods provided herein can also be used to increase the chances that embryonic stem cells which have been genetically engineered to contain one or more transgenes will be incorporated into the germ line of transgenic animals.

Among the several aspects of the invention, therefore, is provided a method for enhancing the generation and/or proliferation of primordial germ cells comprising, obtaining pluripotent cells from a mammalian embryo, and contacting the pluripotent cells in vitro with a primordial germ cell enhancing amount of at least one 60A class BMP protein and at least one DPP class BMP protein for a time sufficient to enhance primordial germ cell formation and/or proliferation.

In another aspect is provided, a method for introducing at least one transgene into the germ line of an animal comprising, obtaining pluripotent cells from a mammalian embryo and if the pluripotent cells do not contain a transgene of interest, introducing at least one transgene by any suitable method known in the art. The transgenic pluripotent cells are then contacted in vitro with a primordial germ cell enhancing amount of at least one 60A class BMP protein and at least one DPP class BMP protein for a time sufficient to enhance primordial germ cell formation and/or proliferation, and transplanting the transgenic pluripotent cells treated with the BMP proteins into the proximal epiblast of a mammalian embryo.

In still another aspect is provided a method for treating sterility in a mammal comprising, obtaining pluripotent cells from a mammalian animal; contacting the pluripotent cells in vitro with a primordial germ cell enhancing amount of at least one 60A class BMP and at least one DPP class BMP protein; and transplanting the pluripotent cells treated with the BMP proteins into the seminiferous tubules of a sterile mammal.

### BRIEF DESCRIPTION OF THE DRAWINGS

These and other features, aspects, and advantages of the present invention will become better understood with regard to the following description, appended claims and accompanying figures where:

FIG. 1 shows the DNA constructs used for COS cell transfections. IRES, is the parental vector (pIRES) which contains a strong cytomegalovirus immediate early promoter (CMV promoter) followed by an intervening sequence (IVS), multiple cloning site A (MCS A), internal ribosome entry site (IRES) of the encephalomyocarditis virus, multiple cloning site B (MCS B), and the SV40 polyadenylation signal. pIRES also has a neomycin resistance gene to allow selection. Bmp4 is a pIRES construct containing Bmp4 in MCS A. Bmp8b is a pIRES construct containing Bmp8b in MCS B. Bmp4/Bmp8b is a pIRES construct containing Bmp4 and Bmp8b in MCS A and MCS B, respectively.

FIG. 2 shows the simultaneous requirement for both BMP4 and BMP8b to enhance the generation of primordial germ cells from pluripotent embryonic cells. The number in parenthesis is the number of epiblasts in which primordial germ cells were determined. Cos7-IRES=vector control cells, Cos7-Bmp4=cells expressing Bmp4, Cos7-Bmp8b=cells expressing Bmp8b, Cos7-Bmp4/Bmp8b=cells expressing both Bmp4 and Bmp8b, Cos7-Bmp4/Cos7-Bmp8b=a mixture of cells expressing Bmp4 and cells expressing Bmp8b.

FIG. 3A shows the percentage of Bmp8b−/− epiblast masses with primordial germ cells when co-cultured with control cells (IRES) or a combination of cells expressing Bmp4 and cells expressing Bmp8b.

FIG. 3B shows the number of primordial germ cells per epiblast mass containing PGCs when co-cultured with control cells (IRES) or a combination of cells expressing Bmp4 and cells expressing Bmp8b.

FIG. 4A shows the number of primordial germ cells (PGCs) at the neuroplate (NP) and head fold (HF) stages in wild-type (WT), heterozygous (Bmp2+/−) and homozygous (Bmp2−/−) embryos.

FIG. 4B shows regression analysis of primordial germ cell (PGC) number versus somite number for wild-type (WT)(solid circles, upper line), Bmp2+/− heterozygotes (open circles, middle line) and Bmp2−/− homozygotes (triangles, lower line)

FIG. 5 shows linear regression analysis of primordial germ cell (PGC) number versus somite number for wild-type (solid circles, upper heavy line), Bmp2 heterozygotes (open circles, upper light line), Bmp4 heterozygotes (open circles, low light line) and Bmp2/Bmp4 double heterozygotes (squares, lower heavy line).

### ABBREVIATIONS AND DEFINITIONS

PGC=primordial germ cell

BMP2 refers to bone morphogenetic protein 2 and variants thereof.

BMP4 refers to bone morphogenetic protein 4 and variants thereof.

BMP8B refers to bone morphogenetic protein 8B and variants thereof.

As used herein, the terms “generating” or “generation” when used in reference to primordial germ cells refers to an increase in the number of primordial germ cells whether by the differentiation of cells to create new primordial germ cells or due to proliferation by cell division of existing primordial germ cells.

As used herein in reference to embryonic cells, the term “pluripotent” means capable of differentiating into more than one alternative type of mature cell.

### DETAILED DESCRIPTION

The following detailed description is provided to aid those skilled in the art in practicing the present invention. Even so, this detailed description should not be construed to unduly limit the present invention as modifications and variations in the embodiments discussed herein can be made by those of ordinary skill in the art without departing from the spirit or scope of the present inventive discovery.

All publications, patents, patent applications and other references cited in this application are herein incorporated by reference in their entirety as if each individual publication, patent, patent application or other reference were specifically and individually indicated to be incorporated by reference.

A method for enhancing the generation of primordial germ cells from pluripotent mammalian embryonic cells has been developed. The method involves contacting the pluripotent cells with a combination of at least one 60A class BMP protein and at least one DPP class BMP protein. The applicants have demonstrated that by using the methods described herein, that it is possible to enhance the number of primordial germs cells from pluripotent cells as indicated by staining for alkaline phosphotase in the cytoplasmic membrane and Golgi apparatus which is characteristic of PGCs.

The source of the pluripotent mammalian embryonic cells is thought to be only broadly critical to practicing the invention. In one preferred embodiment, the pluripotent cells are obtained from the epiblast, also known as the primitive ectoderm, of a mammalian embryo. No particular site within the epiblast is preferred. In one embodiment, epiblast cells are obtained from mice at embryonic day 6.25 (E 6.25) where the day of the vaginal plug (mating) is designated E 0.5. Methods for the isolation of epiblast cells are well known in the art. For example, Hogan et al. (Manipulating the Mouse Embryo, Cold Spring Harbor Laboratory Press, 1994, pp 151-165) describes methods for the isolation of epiblast cells from mice. Because of the high degree of similarity in early embryonic development in mammals, such methods can be easily adapted for use in other mammals by those of ordinary skill in the art without undue experimentation. The major differences between species is the time at which the various developmental stages are reached. Since these times are known in the art and can be found in standard texts and treatises on developmental biology, for example, Cupps, ed., Reproduction in Domestic Animals, 4th ed., Academic Press, 1990; and Gilbert, Developmental Biology, Sinauer, 1988, the skilled technician can readily determine the proper time after mating to obtain epiblast tissue. Also, due to the high degree of homology between mammalian species, it is not necessary that the BMPs and pluripotent embryonic cells originate from the same species.

Alternatively, pluripotent cells can be obtained from embryonic stem cells. Embryonic stem cells are pluripotent cells derived from the inner cell mass of embryos. Under proper culture conditions, these cells can be maintained in a pluripotent state in vitro. When desired, embryonic stem cells can be induced to undergo differentiation by, for example, alteration of the culture conditions or by transplantation to an embryo or animal. Methods for the production of embryonic stem cells in a variety of mammals are known in the art and can be found for example in U.S. Pat. No. 5,985,659 (mice), Hogan et al., (Manipulating the Mouse Embryo, 2nd ed., Cold Spring Harbor Laboratory Press, 1994; mouse), Evans and Kaufman (Nature 292:154, 1981, mice), Anderson (Anim. Biotechnol. 3:165-175, 1992; livestock) U.S. Pat. No. 6,107,543 (cattle), Hassan-Hauser et al. (Reprod. Dom. Anim. 25:22-32, 1990, cattle) U.S. Pat. No. 6,103,523 (rabbits), U.S. Pat. No. 6,090,622 (humans), U.S. Pat. No. 5,994,619 (cattle and swine), Evans et al. (Theriogenology, 33:125, 1990; cattle and swine), U.S. Pat. Nos. 5,942,435 and 5,523,226 (swine), Notarianni et al (Proc. 4th World Cong. Genet. Appl. Livestock Prod. 1991, pp.58-64; sheep and swine), and Doetschman et al (Develop. Biol. 127:224, 1988, hamster).

In one embodiment, embryonic stem cells are induced to form primitive ectoderm prior to use in the method of the present invention. Embryonic stem cells can be induced to form primitive ectoderm by transplantation into embryos at the blastocyst stage or greater using methods that are well known in the art of developmental biology. Embryonic stem cells can also be induced by alteration of the culture conditions such that the embryonic stem cells form embryoid bodies as is well known in the art.

In another alternative, pluripotent cells are obtained from inner cell masses (ICMs) isolated from late blastocyst stage embryos. Methods for obtaining ICM cells are well known in the art and include manual dissection and immunosurgery (See Hogan et al, Manipulating the Mouse Embryo, 2nd ed., Cold Spring Harbor Laboratory Press, 1994; Solter and Knowles, Proc. Natl. Acad. Sci. USA 72:5099-5102, 1975).

Once obtained, the pluripotent embryonic cells are treated with at least one member of the BMP superfamily. Known members of the BMP superfamily and their relationship have been previously described (Hogan, Genes Develop., 10:1580-1594, 1996) and include GDF10, BMP3/osteogenin, BMP9, Dorsalin 1 (chicken), BMP10, Vgr2/GDF3, GDF5 (brachypodism), BMP13/GDF6, BMP12/GDF7, BMP5 (short ear), BMP6/Vgr1, BMP7/OP1, BMP8A/OP2, BMP8B, 60A (Drosophila), BMP2, BMP4, DPP (Drosophila), Vg1 (Xenopus), Univin (sea urchin), GDF1, Screw (Drosophlia), and Nodal. In one embodiment pluripotent embryonic cells are treated with combination of at least one 60A class BMP protein and at least one DPP class BMP protein. A preferred 60A class BMP protein is BMP8B. Preferred DPP class BMP proteins include BMP2 and BMP4. The amino acid sequences for BMP proteins from a variety of species are known in the art and can be found in well known databases such as those linked to the National Center for Biotechnology Information http://www.ncbi.nlm.nih.gov/ and the European Bioinformatics Institute http://www.ebi.ac.uk/, both herein incorporated by reference.

Those of ordinary skill in the art are aware that modifications in the amino acid sequence of a peptide, polypeptide, or protein can result in equivalent, or possibly improved, second generation peptides, etc., that display substantially equivalent or superior biological activity when compared to the original amino acid sequence. In addition to known sequences, the present invention includes variants and fragments of BMPs useful in the present invention. Fragment or variants can include amino acid insertions, deletions, substitutions, truncations, fusions, shuffling of subunit sequences, and the like, provided that the protein variants or fragments produced by such modifications have substantially the same biological activity as the naturally occurring counterpart proteins. By substantially the same biological activity is meant that the modified protein when combined with its appropriate counterpart, e.g. a DPP class BMP protein for a 60A class protein, will enhance the generation of primordial germ cells. By enhance is meant that the number of primordial germ cells formed is greater than the number that would have been formed without the use of the method of the present invention. Thus, enhancement includes the formation of new primordial germ cells as well as an increase in number. The generation of primordial germ cells can be determined by well known methods such as alkaline phosphatase staining which is detailed in references cited herein and used in the examples that follow.

One factor that can be considered in making such changes is the hydropathic index of amino acids. The importance of the hydropathic amino acid index in conferring interactive biological function on a protein has been discussed by Kyte and Doolittle (J. Mol. Biol., 157: 105-132, 1982). It is accepted that the relative hydropathic character of amino acids contributes to the secondary structure of the resultant protein. This, in turn, affects the interaction of the protein with molecules such as enzymes, substrates, receptors, DNA, antibodies, antigens, etc.

Based on its hydrophobicity and charge characteristics, each amino acid has been assigned a hydropathic index as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate/glutamine/aspartate/asparagine (−3.5); lysine (−3.9); and arginine (−4.5).

As is known in the art, certain amino acids in a peptide or protein can be substituted for other amino acids having a similar hydropathic index or score and produce a resultant peptide or protein having similar biological activity, i.e., which still retains biological functionality. In making such changes, it is preferable that amino acids having hydropathic indices within ±2 are substituted for one another. More preferred substitutions are those wherein the amino acids have hydropathic indices within ±1. Most preferred substitutions are those wherein the amino acids have hydropathic indices within ±0.5.

Like amino acids can also be substituted on the basis of hydrophilicity. U.S. Pat. No. 4,554,101 discloses that the greatest local average hydrophilicity of a protein, as governed by the hydrophilicity of its adjacent amino acids, correlates with a biological property of the protein. The following hydrophilicity values have been assigned to amino acids: arginine/lysine (+3.0); aspartate/glutamate (+3.0±1); serine (+0.3); asparagine/glutamine (+0.2); glycine (0); threonine (−0.4); proline (−0.5±1); alanine/histidine (−0.5); cysteine (−1.0); methionine (−1.3); valine (−1.5); leucine/isoleucine (−1.8); tyrosine (−2.3); phenylalanine (−2.5); and tryptophan (−3.4). Thus, one amino acid in a peptide, polypeptide, or protein can be substituted by another amino acid having a similar hydrophilicity score and still produce a resultant protein having similar biological activity, i.e., still retaining correct biological function or activity. In making such changes, amino acids having hydropathic indices within ±2 are preferably substituted for one another, those within ±1 are more preferred, and those within ±0.5 are most preferred.

As outlined above, amino acid substitutions in the peptides of the present invention can be based on the relative similarity of the amino acid side-chain substituent, for example, their hydrophobicity, hydrophilicity, charge, size, etc. Exemplary substitutions that take various of the foregoing characteristics into consideration in order to produce conservative amino acid changes resulting in silent changes within the present peptides, etc., can be selected from other members of the class to which the naturally occurring amino acid belongs. Amino acids can be divided into the following four groups: (1) acidic amino acids; (2) basic amino acids; (3) neutral polar amino acids; and (4) neutral non-polar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral non-polar amino acids such as alanine, leucine, isoleucine, valine, proline, phenylalanine, tryptophan, and methionine. It should be noted that changes which are not expected to be advantageous can also be useful if these result in the production of functional sequences.

The enhancement of primordial germ cell generation by the method of the present invention is achieved by contacting pluripotent embryonic cells with a combination of at least one 60A class BMP protein, preferably BMP8B, and at least one DPP class BMP protein, preferably BMP2 or BMP4. A variety of methods can be used to contact the pluripotent embryonic cells with the BMP proteins in accordance with the present invention. In one embodiment, at least one 60A class BMP protein and at least one DPP class BMP protein are added to culture medium containing pluripotent embryonic cells.

In another embodiment, a co-culture system is used. For example, the pluripotent cells can be co-cultured in the presence of cells that secrete one or more 60A class BMP proteins and one or more DPP class BMP proteins into the culture medium, such as cells that secrete BMP8B and BMP2, cells that secrete BMP8B and BMP4, cells that secrete BMP8B, BMP2 and BMP4, or any combination of the preceding cells. Alternatively, the pluripotent cells can be co-cultured with a combination of cells each of which secrete a different BMP protein or combination of BMP proteins. Cells are combined so that at least one 60A class BMP protein and at least one DPP class BMP protein are secreted into the medium. Examples of suitable combinations include, cells secreting BMP8B in combination with cells secreting BMP2, BMP4 or a combination of BMP2 and BMP4. Other suitable combinations of cells will be apparent to those of ordinary skill in the art and are within the scope of the present invention.

When co-culture systems are used, the cells expressing the BMPs are preferably treated to inhibit cell division. Any method that prevents or significantly inhibits cell division while allowing the production and secretion of BMPs can be used. Non-limiting examples, of suitable methods include, treatment with mitomycin C and irradiation. In one preferred embodiment, cell division is inhibited by mitomycin C treatment.

In another embodiment, conditioned medium can be used. Conditioned medium is medium which has previously been used to culture cells secreting at least one 60A class BMP protein and at least one DPP class protein so that the culture medium contains primordial germ cell generation enhancing amounts of the BMP proteins. Any of the combination of cells discussed in relation to co-culture systems can be used to produce conditioned medium.

The cells used to secrete BMPs into culture medium can be cells that naturally secrete BMP proteins or cells that have been genetically modified using known molecular biology techniques to secrete BMPs. Any known method for the production of transgenic cells can be used so long as the resulting cells secrete BMPs into the medium. Production of genetically modified cells by well established methods such as those described in Sambrook et al., Molecular Cloning, 2nd ed., Cold Spring Harbor Laboratory Press, 1989 and Ausubel et al., Short Protocols in Molecular Biology, 3rd ed., Wiley & Sons, 1995, is routine in the art. Suitable methods for the production of transgenic cells include, but are not limited to, calcium phosphate transfection, DEAE-dextran mediated transfection, Polybrene, protoplast fusion, liposomes, direct microinjection into the nuclei, scrape loading, and electroporation. In one preferred embodiment, transfection is by electroporation.

The form of the BMPs, either as homodimers or heterodimers, is not thought to be critical to the practice of the invention. As discussed previously, BMP proteins are produced as dimers. Because of the high degree of sequence homology between BMP proteins, cells secreting more than one BMP are expected to produce a combination of homodimeric and heterodimeric BMP proteins. As is shown in the examples below, cells secreting more than one form of BMP protein are fully capable of enhancing primordial germ cell generation.

The amount of BMP protein required is thought to be only broadly critical to the practice of the invention. The exact concentration will vary with the species in question and the exact BMPs used. In instances where the exposure of the pluripotent embryonic cells to the BMPs is by co-culture or conditioned medium, the final concentration of BMPs in the medium may be unknown. The amount of BMP protein needed, the number of cells for co-culture, or the number of cells and time required to condition medium can be determined empirically by those of ordinary skill in the art without undue experimentation. The amount of BMPs used should be sufficient to enhance the generation of primordial germ cells. Methods for the detection and identification or primordial germ cells are well known in the art. For example, in one embodiment, the presence of primordial germ cells is determined by staining for alkaline phosphatase as described by Ginsburg et al., (Development, 100:521-528, 1990); Lawson et al., (Genes Devel. 13:424-436,1999), Ying et al., (Molec. Endocrinol. 14:1053-1063, 2000); and described in the examples that follow.

Likewise, the time during which the pluripotent cells are exposed to the BMP proteins is thought to be only broadly critical, but should be sufficient to enhance generation of primordial germ cells. It will be recognized by those skilled in the art, that the exact time required to enhance primordial germ cell generation will vary with such factors as the amount and type of BMPs; the method used to contact the pluripotent cells with the BMP proteins, for example, co-culture or conditioned medium; and the species of embryo. The minimum time required to enhance generation of primordial germ cells can be empirically determined by those of ordinary skill in the art using the methods described above and in the following examples. In general, the time should be between about 1 and 120 hours, preferably between about 24 and 96 hours, and more preferably about 72 hours.

In one embodiment, the method of the present invention is used to introduce a transgene into the germ line of an animal. The presence of transgenes in the germ cells of transgenic animals is of critical importance, since it allows for transfer of the transgene to the progeny of the transgenic animal. In this embodiment, pluripotent cells containing the gene or genes of interest are obtained. If the pluripotent cells do not contain the gene(s) of interest the cells can be genetically modified using well known methods such as those discussed previously to introduce the genes into the cells. Once pluripotent cells containing the gene or gene of interest are obtained, the cells are incubated with at least one 60A class BMP protein, preferably BMP8B, and at least one DPP class BMP protein, preferably BMP 2 or BMP4, using any of the methods previously described.

Following treatment, the pluripotent cells are then transferred into the epiblast, preferably the proximal epiblast, of a developing recipient embryo. It is preferred that the transplanted cells be of the same sex as the recipient embryo, for example XX (female) pluripotent cells transplanted into XX embryos. The cells transplanted can have originally been obtained from the same or different embryo. If the transplanted cells are from a different embryo, the resulting embryo will be chimeric. The production of animals with chimeric germ lines is especially useful since genetic markers, for example coat color, can be exploited to readily determine if the offspring carry the transgene. In another embodiment, the transplanted cells are from a different, but closely related species, in order to produce an interspecific chimeric embryo. Interspecific chimeras have been produced between a number of species and are well known in the art. In one embodiment, the cells are transplanted into an embryo in utero and the embryo is allowed to develop to term. Because the number of primordial germ cells produced by the cells has been enhanced, the likelihood that cells containing the gene(s) of interest will develop into germ cells is likewise enhanced.

In another embodiment, pluripotent embryonic cells treated with a combination of a 60A class BMP protein and a DPP class BMP protein are used to treat sterility in an animal. In one preferred embodiment, the animal is a male animal. In this embodiment, pluripotent embryonic cells, preferably of the same sex as the animal to be treated, are obtained and incubated with a combination of at least one 60A class BMP protein and at least one DPP class BMP protein to enhance primordial germ cell generation as has been described previously. In one embodiment, once the cells have been treated with the BMP proteins, they are transferred into the lumen of the seminiferous tubules of the testes. Introduction of cells into the seminiferous tubules can be accomplished by the use of microsurgical techniques. Once in the lumen, the cells can colonize the immature germ cells lining the seminiferous tubules where they can enter into spermatogenesis and develop into mature spermatozoa.

It will be apparent to those of ordinary skill in the art that if the absence of, or reduced number of, primordial germs cells is due to an absence of only one class of BMP protein, then it may be necessary to supply only the missing class of BMP protein. For example, and without limitation, if pluripotent embryonic cells are obtained from an animal that is Bmp8b−/− but wild type for the corresponding Dpp gene (e.g. Bmp4+/+), contacting the pluripotent cells with BMP8B or exposing the pluripotent cells to cells expressing Bmp8b or conditioned medium containing BMP8B is sufficient to enhance primordial germ cell generation.

Because its development has been extensively studied, many of the examples herein relate to the mouse. However, as discussed previously, BMP proteins are widely dispersed across the animal kingdom. In addition, although differences exist in the embryonic development of mammals, it is widely recognized that many similarities exist, especially in terms of early embryonic development. In particular, it is thought that the mechanism by which primordial germ cells develop is conserved across mammals. Thus, the present invention is not limited in its application to mice, but rather, is applicable to all mammalian species, including but not limited to humans, livestock such as cows, sheep, goats, pigs, horses, etc., domestic pets such as cats and dogs, rodents, exotic mammals such as zoo animals, etc.

### EXAMPLES

The following examples are intended to provide illustrations of the application of the present invention. The following examples are not intended to completely define or otherwise limit the scope of the invention.

### Example 1

### Generation of BMP Expressing COS Cells

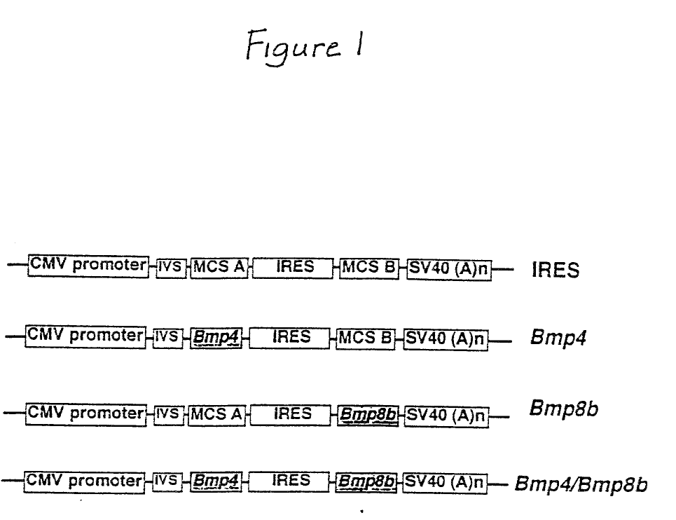

The fill length coding region of human Bmp4 cDNA and murine Bmp8b cDNA were inserted into multiple cloning site A (MCS A) and MCS B, respectively, of the pIRES vector (Clontech, Palo Alto, Calif.) using standard techniques known in molecular biology and detailed, for example, in Sambrook et al., Molecular Cloning, 2nd ed., Cold Spring Harbor Laboratory Press, 1989 (FIG. 1). About 5-10 μg of DNA was used to transfect 10⁷ exponentially-growing COS cells in 1 ml of ice-cold phosphate buffered saline (PBS) by electroporation using a Gene Pulser II system (Bio Rad Laboratories, Hercules, Calif.). For electroporation, the capacitance was set a 960 μF and the voltage was set at 250 volts. Transfected COS cells were grown in standard culture medium (DMEM with 4 mM/l glutamine, 4.5 g/l glucose 1.5 g/l sodium bicarbonate, 100 IU/ml penicillin, 100 μg/ml streptomycin and 10% fetal bovine serum (FBS)) for 24 hours. The medium was then supplemented with 360 μg/ml G418. G418 selection was conducted for 14 days to obtain a mixture of resistant clones.

### Example 2

### Isolation and Culture of Pluripotent Cells

Three different genetic backgrounds of laboratory mice were used, including the outbred ICR strain, 87.5% C57BL/6 strain, and the F1 generation of a C57BL/6 and 129SvEv cross. All mice were housed in a controlled environment of a 12 hour light/dark cycle, 40-50% humidity, and 70-75° F. Cycling females were caged with males for mating. Noon of the day of mating (vaginal plug present) was designated at embryonic day 0.5 (E 0.5). All embryos were collected and dissected for culture on E 6.25. Once collected, the embryos were stored on ice and the extraembryonic regions were removed by tungsten needles as describe by Hogan et al., Manipulating the Mouse Embryo, 2nd ed., Cold Spring Harbor Laboratory Press, 1994, pp 151-165. The embryonic portions containing epiblast cells and endoderm were then incubated in calcium/magnesium-free Tyrode Ringer's saline containing 2.5% pancreatin and 0.5% trypsin for 10-15 minutes at 4° C. Endoderm was removed by gentle pipetting Up and down a few times with Pasteur pipette.

### Example 3

### Co-culture of Pluripotent Embryonic Cells with BMP Secreting COS Cells

COS cells, genetically modified as described in Example 1, were grown on 35×10 mm petri dishes to 75%-100% confluence. Combinations tested included COS cells transfected with Bmp8b alone, Bmp4 alone, Bmp8b and Bmp4, and a combination of cells transfected with Bmp8b alone and cells transfected with Bmp4 alone. All COS cells were inactivated in culture medium containing 5 μg/ml mitomycin C for 3 hours, washed twice with PBS, and then maintained in standard culture medium or standard culture medium containing 15% FBS. Epiblast cell masses isolated as described in Example 2, were added to the petri dishes containing the COS cells and co-cultured for 72 hours. After 72 hours, the epiblast masses were fixed in 4% formaldehyde-PBS and stained for alkaline phosphatase activity to detect primordial germ cells using the method described in Ginsberg et al., (Development, 110:521-528, 1990); Lawson et al, (Genes Devel. 13:424-436, 19990; and Ying et al., (Molec. Endocrinol., 14:1053-1063, 2000).

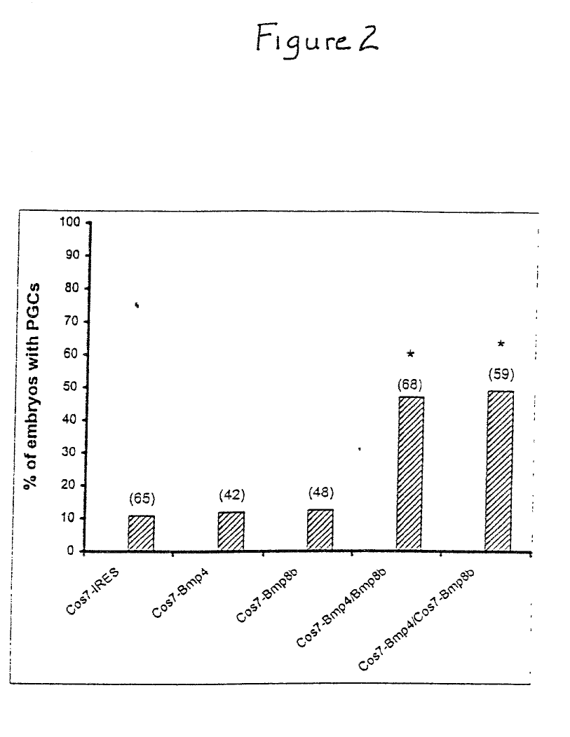

The results are shown in FIG. 2. When pluripotent cells from the epiblast were co-cultured with COS cells transfected with vector alone (control), COS cells transfected with Bmp4 expressing vector, or COS cells transfected with Bmp8b expressing vector, no significant difference (p>0.05) was observed in the percentage cell masses containing primordial germ cells. In contrast, when epiblast cell masses were co-cultured with COS cells transfected with both Bmp4 and Bmp8b expressing vector or a combination of COS cells transfected with Bmp4 expressing vector and COS cells transfected with Bmp8b expressing vector, a significantly higher percentage (p<0.0001) of cell masses contained primordial germ cells. No significant difference in the percentage of cell masses containing primordial germ cells was seen between epiblast cell masses co-cultured with cells expressing both Bmp4 and Bmp8b or a mixture of Bmp4 and Bmp8b expressing cells.

As discussed previously, mature BMPs are a dimer. COS cells transfected with Bmp4 and Bmp8b expressing vector are, therefore, expected to secrete a mixture of BMP4 homodimer, BMP8b homodimer and BMP4/BMP8b heterodimer. In contrast, in co-culture systems utilizing a combination of COS cells transfected with Bmp4 expressing vector and COS cells transfected with Bmp8b expressing vector, only homodimers will be present. The results clearly show that a combination of BMP4 and BMP8b homodimers is sufficient to enhance primordial germ cell generation. The finding that there was no significant difference in percentage of cell masses containing primordial germ cells indicates that BMP4/BMP8b heterodimers also are active in the generation of primordial germ cells.

In order to further validate that BMP4 and BMP8b homodimers are capable of enhancing primordial germ cell generation in pluripotent cells, epiblast cells from Bmp8b −/− (null mutant) embryos of 87.5% C57BL/6 were isolated as described in Example 2. Epiblast cells were co-cultured with COS cells transfected with vector alone (control) or a combination of COS cells transfected with Bmp4 expressing vector and Cos cells Bmp8b expressing vector as described above.

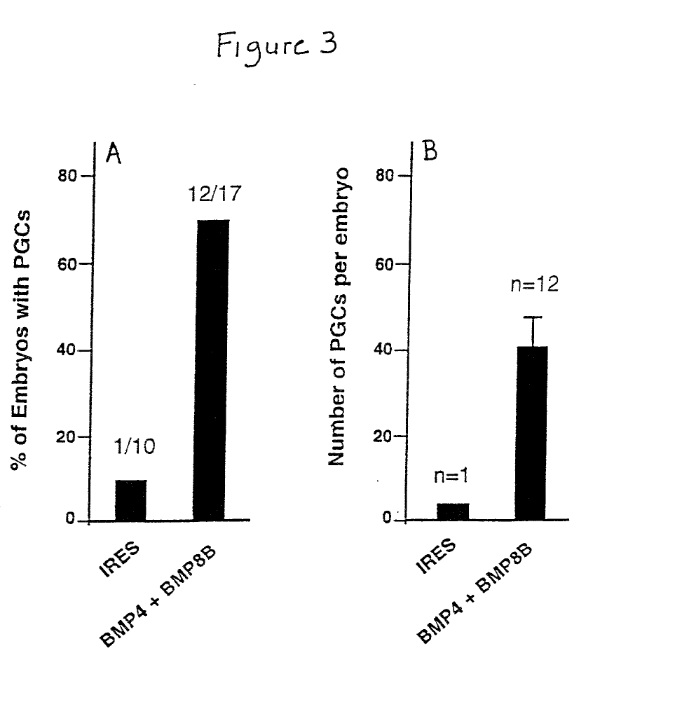

As can be seen in FIG. 3A, only one out of 10 Bmp8b−/− embryos co-cultured with control COS cells contained primordial germ cells, while 12 of 17 embryos co-cultured with a combination of COS cells expressing Bmp4 and Bmp8b contained primordial germ cells. The number of primordial germ cells present in embryo containing PGCs is shown in FIG. 3B. Four primordial germ cells were found in the one embryo in which PGCs were detected following co-culture with control COS cells. In contrast, an average of 40.3±6.0 primordial germ cells were found in embryos co-cultured with a combination of COS cells transfected with Bmp4 and COS cells transfected with Bmp8b. Taken in combination with the results shown in FIG. 2, these results show that a combination of BMP4 and BMP8b homodimers are not only capable of enhancing primordial germ cell formation in wild-type embryos, but is also capable of inducing primordial germ cell formation in Bmp8b−/− embryos which would be sterile due to a lack of primordial germ cells.

### Example 4

### Production of Bmp2−/− Mice

Mice heterozygous for a known Bmp2 mutation (Zhang and Bradley, Development 124:3157-3165, 1996) were backcrossed with C57BL/6 inbred females to generate N1 and N2 heterozygotes. N2 male and female heterozygotes were intercrossed to produce null mutants for primordial germ cell analysis. Bmp4 and Bmp8b mutants used have been previously described (Winnier et al., Genes Devel. 9:2105-2116, 1995; Zhao et al., Genes Devel. 10:1657-1669, 1996) and were maintained on a mixed (129/SvEv×Black Swiss) or C57BL/6 genetic backgrounds.

### Example 5

### PGC Staining and Counting in Bmp2−/− Embryos

To count primordial germ cells, whole-mount alkaline phosphatase staining was used as previously described (Ginsburg et al., Development 110:521-528, 1990, Lawson et al, Genes Devel. 13:424-436, 1999). Briefly, embryos at E 7.25 to E 9.5 were collected and fixed in freshly prepared 4% paraformaldehyde in PBS for 2 to 3 hours. The embryos were further dissected to remove the trophoblast, but both the amnion and yolk sac were left attached to the embryos. Embryos were then stained with freshly prepared α-naphthyl phosphate/Fast red TR (Sigma Chemical Co., St. Louis, Mo.,) for 15 to 20 minutes at room temperature. After somite number was counted, the embryos were cut to give anterior and posterior halves. The embryo pieces containing primordial germ cells were mounted on a slide in 70% glycerol under a coverslip. Primordial germ cells were counted with a microscope (400×magnification).

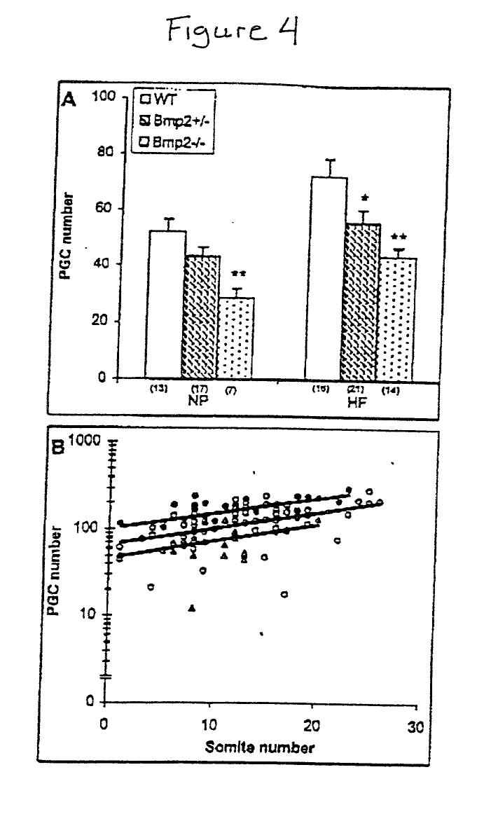

At the late-streak stage, recognizable primordial germ cells were found in 50% of Bmp2 homozygous embryos (n=12), which was not statistically different from the wild-type embryos (64.3%, n=14)p >0.05). To address whether the mutation of Bmp2 reduces primordial germ cell number, the number of PGCs present at different developmental stages was determined. As shown in FIG. 4A, at the neural plate and headfold stages, the number of primordial germ cells in Bmp2 homozygotes was 21.6±3.1 and 43.8±3.0, respectively. This was significantly smaller than the number of primordial germ cells found in wild type embryos (52.1±4.4 and 72.8±6.3, p<0.01).

Regression analysis of primordial germ cells on somite number showed that the number of primordial germ cells in homozygous mutants (Y=1.670+0.0196X, n=26) was consistently smaller than the number in the wild type (Y=2.007+0.0177X, n=28; p<0.001) and heterozygotes (Y=1.823+0.0188X, n=64; p<0.01), but the slopes of the regression lines were similar (FIG. 4B). These results suggest that the major role of BMP2 is not in primordial germ cell proliferation and/or survival, but in the formation of primordial germ cells.

### Example 6

### Additive Effect of Bmp2 and Bmp4

BMP2 and BMP4 are both members of the DPP class of BMPs. Functionally, BMP2 exhibits many of the same activities as BMP4 (Coucouvanis and Martin, Development 126:535-546, 1999; Furtua et al., Development 124:2203-2212, 1977; Hemmati-Brivanlou and Thomsen, Develop. Genet. 17-78-89, 1995; Suzuki et al., Develop. Biol. 189:112-122, 1997; Yokouchi et al, Development, 122:3725-3734, 1996). To determine if Bmp2 and Bmp4 have an additive effect on primordial germ cell generation, double heterozygotes were produced by crossing Bmp2 heterozygotes (129SvEv×C57BL/6) with Bmp4 heterozygotes (129SvEv×Black Swiss). Embryos were processed, stained and primordial germ cells counted as described in Example 5.

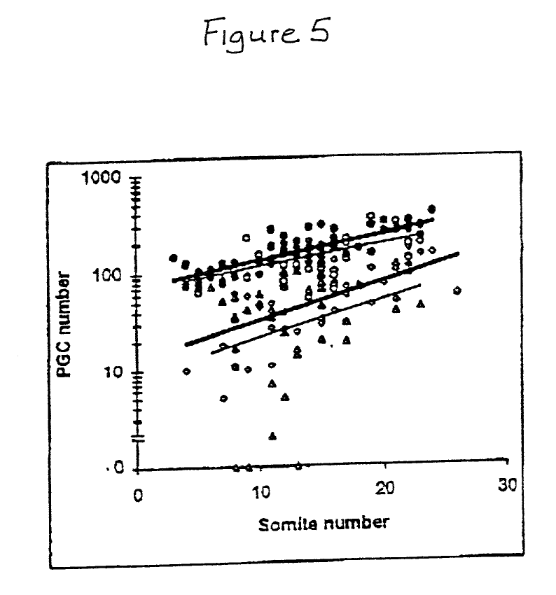

Regression analysis of primordial germ cells versus somite number showed that there was no significant difference in primordial germ cell number between wild-type (Y=1.871+0.0259X, n=60) and Bmp2 heterozygotes (y=1.841+0.0223X, n=53) on this mixed genetic background (FIG. 5, p>0.05). The number of primordial germ cells in Bmp4 heterozygotes (Y=1.249+0.0322, n=44) and Bmp2/Bmp4 double heterozygotes (Y=1.122 +0.0276, n=48), however, were significantly smaller (p<0.001) than the number in wild type and Bmp2 heterozygotes. Moreover, primordial germ cell number in Bmp2 and Bmp4 double heterozygotes was further reduced in comparison to Bmp4 heterozygotes alone (p<0.05), and four embryos in the former groups completely lacked primordial germ cells. These results indicate that Bmp2 and Bmp4 have an additive effect on primordial germ cell formation.

### CONCLUSION

In light of the detailed description of the invention and the examples presented above, it can be appreciated that the several aspects of the invention are achieved.

It is to be understood that the present invention has been described in detail by way of illustration and example in order to acquaint others skilled in the art with the invention, its principles, and its practical application. Particular formulations and processes of the present invention are not limited to the descriptions of the specific embodiments presented, but rather the descriptions and examples should be viewed in terms of the claims that follow and their equivalents. While some of the examples and descriptions above include some conclusions about the way the invention may function, the inventors do not intend to be bound by those conclusions and functions, but put them forth only as possible explanations.

It is to be further understood that the specific embodiments of the present invention as set forth are not intended as being exhaustive or limiting of the invention, and that many alternatives, modifications, and variations will be apparent to those of ordinary skill in the art in light of the foregoing examples and detailed description. Accordingly, this invention is intended to embrace all such alternatives, modifications, and variations that fall within the spirit and scope of the following claims.

What is claimed is:

1. A method for enhancing the number of primordial germ cells comprising:
a) obtaining pluripotent cells from a mammalian embryo; and
b) contacting said pluripotent cells in vitro with a primordial germ cell number enhancing amount of at least one 60A class BMP protein and at least one DPP class BMP protein for a time sufficient to enhance primordial germ cell generation.

2. The method of claim 1, wherein said at least one 60A class BMP protein is BMP8B.

3. The method of claim 1 wherein said at least one DPP class BMP protein is selected from the group consisting of BMP2 and BMP4.

4. The method of claim 1, wherein said pluripotent cells are epiblast cells.

5. The method of claim 1, wherein said pluripotent cells are inner cell mass cells.

6. The method of claim 5, further comprising inducing the formation of epiblast cells from said inner cell mass cells.

7. The method of claim 1, wherein said pluripotent cells are embryonic stem cells.

8. The method of claim 7, further comprising inducing the formation of epiblast cells from said embryonic stem cells.

9. The method of claim 1, wherein said contacting of said pluripotent cells with said BMP proteins comprises co-culturing said pluripotent cells with cells selected from the group consisting of:
a) cells naturally secreting BMP4 and BMP8B;
b) cells naturally secreting BMP2 and BMP8B;
c) cells naturally secreting BMP2, BMP4 and BMP8B;
d) a combination of cells naturally secreting BMP4 and cells naturally secreting BMP8B;
e) a combination of cells naturally secreting BMP2 and cells naturally secreting BMP8B;
f) a combination of cells naturally secreting BMP2, cells naturally secreting BMP4, and cells naturally secreting BMP8B;
g) transgenic cells secreting BMP4 and BMP8B;
h) transgenic cells secreting BMP2 and BMP8B;
i) transgenic cells secreting BMP2, BMP4 and BMP8B;
j) a combination of transgenic cells secreting BMP4 and transgenic cells secreting BMP8B;
k) a combination of transgenic cells secreting BMP2 and transgenic cells secreting BMP8B;
l) a combination of transgenic cells secreting BMP2, transgenic cells secreting BMP4, and transgenic cells secreting BMP8B; and
m) a combination of any of the above.

10. The method of claim 1, wherein said contacting of said pluripotent cells with said BMP proteins comprises addition of a primordial germ cell enhancing amount of BMP8B, and BMP4, BMP2, or a combination of BMP2 and BMP4 to the medium containing said pluripotent cells.

11. The method of claim 1, wherein said contacting of said pluripotent cells with said BMP proteins comprises contacting said pluripotent cells with medium previously used to culture cells selected from the group consisting of:
a) cells naturally secreting BMP4 and BMP8B;
b) cells naturally secreting BMP2 and BMP8B;
c) cells naturally secreting BMP 2, BMP4 and BMP8B;
d) a combination of cells naturally secreting BMP4 and cells naturally secreting BMP8B;
e) a combination of cells naturally secreting BMP2 and cells naturally secreting BMP8B;
f) a combination of cells naturally secreting BMP2, cells naturally secreting BMP4, and cells naturally secreting BMP8B;
g) transgenic cells secreting BMP4 and BMP8B;
h) transgenic cells secreting BMP2 and BMP8B;
i) transgenic cells secreting BMP2, BMP4 and BMP8B;
j) a combination of transgenic cells secreting BMP4 and transgenic cells secreting BMP8B;
k) a combination of transgenic cells secreting BMP2 and transgenic cells secreting BMP8B;
l) a combination of transgenic cells secreting BMP2, transgenic cells secreting BMP4, and transgenic cells secreting BMP8B; and
m) a combination of any of the above.

12. A method for introducing at least one transgene into the germ line of an animal comprising:
a) obtaining pluripotent cells from a mammalian embryo;
b) if said pluripotent cells do not contain at least one transgene of interest, introducing at least one transgene into said pluripotent cells;
c) contacting said transgenic pluripotent cells with a primordial germ cell enhancing amount of at least one 60A class BMP protein and at least one DPP class BMP protein for a time sufficient to induce primordial germ cell generation; and
d) transplanting the pluripotent cells of (c) into the proximal epiblast of a mammalian embryo.

13. The method of claim 12, wherein said at least one 60A class BMP protein is BMP8B.

14. The method of claim 12, wherein said at least one DPP class BMP protein is selected from the group consisting of BMP2 and BMP4.

15. The method of claim 12, further comprising allowing said embryo to develop to term.

16. The method of claim 12, wherein the embryo resulting after (d) is a chimeric embryo.

17. The method of claim 16, wherein said chimeric embryo is an intraspecific chimera.

18. The method of claim 16, wherein said chimeric embryo is an interspecific chimera.

19. The method of claim 12, wherein said pluripotent cells are epiblast cells.

20. The method of claim 12, wherein said pluripotent cells are inner cell mass cells.

21. The method of claim 20, further comprising inducing the formation of epiblast cells from said inner cell mass cells.

22. The method of claim 12, wherein said pluripotent cells are embryonic stem cells.

23. The method of claim 22, further comprising inducing the formation of epiblast cells from said embryonic stem cells.

24. The method of claim 12, wherein said contacting of said pluripotent cells with said BMP proteins comprises co-culturing said pluripotent cells with cells selected from the group consisting of:
a) cells naturally secreting BMP4 and BMP8B;
b) cells naturally secreting BMP2 and BMP8B;
c) cells naturally secreting BMP 2, BMP4 and BMP8B;
d) a combination of cells naturally secreting BMP4 and cells naturally secreting BMP8B;
e) a combination of cells naturally secreting BMP2 and cells naturally secreting BMP8B;
f) a combination of cells naturally secreting BMP2, cells naturally secreting BMP4, and cells naturally secreting BMP8B;
g) transgenic cells secreting BMP4 and BMP8B;
h) transgenic cells secreting BMP2 and BMP8B;
i) transgenic cells secreting BMP2, BMP4 and BMP8B;
j) a combination of transgenic cells secreting BMP4 and transgenic cells secreting BMP8B;
k) a combination of transgenic cells secreting BMP2 and transgenic cells secreting BMP8B;
l) a combination of transgenic cells secreting BMP2, transgenic cells secreting BMP4, and transgenic cells secreting BMP8B; and
m) a combination of any of the above.

25. The method of claim 12, wherein said contacting of said pluripotent cells with said proteins comprises addition of a primordial germ cell enhancing amount of BMP8B, and BMP2, BMP4 or a combination of BMP2 and BMP4 to the medium containing said pluripotent cells.

26. The method of claim 12, wherein said contacting of said pluripotent cells with BMP4 and BMP8B proteins comprises contacting said pluripotent cells with medium previously used to culture cells selected from the group consisting of:
a) cells naturally secreting BMP4 and BMP8B;
b) cells naturally secreting BMP2 and BMP8B;
c) cells naturally secreting BMP 2, BMP4 and BMP8B;
d) a combination of cells naturally secreting BMP4 and cells naturally secreting BMP8B;
e) a combination of cells naturally secreting BMP2 and cells naturally secreting BMP8B;
f) a combination of cells naturally secreting BMP2, cells naturally secreting BMP4, and cells naturally secreting BMP8B;
g) transgenic cells secreting BMP4 and BMP8B;
h) transgenic cells secreting BMP2 and BMP8B;
i) transgenic cells secreting BMP2, BMP4 and BMP8B;
j) a combination of transgenic cells secreting BMP4 and transgenic cells secreting BMP8B;
k) a combination of transgenic cells secreting BMP2 and transgenic cells secreting BMP8B;
l) a combination of transgenic cells secreting BMP2, transgenic cells secreting BMP4, and transgenic cells secreting BMP8B; and
m) a combination of any of the above.

27. A method for treating sterility in a mammal comprising,
a) obtaining pluripotent cells from a mammalian embryo;
b) contacting said pluripotent cells in vitro with a primordial germ cell enhancing amount of at least one 60A class BMP protein and at least one DPP class BMP protein for a time sufficient to enhance primordial germ cell generation; and
c) transplanting said primordial germ cells into the seminiferous tubules of said sterile mammal.

28. The method of claim 27, wherein said at least one 60A class BMP protein is BMP8B.

29. The method of claim 27, wherein said at least one DPP class BMP protein is selected from the group consisting of BMP2 and BMP4.

30. The method of claim 27, wherein said pluripotent cells are epiblast cells.

31. The method of claim 27, wherein said pluripotent cells are inner cell mass cells.

32. The method of claim 31, further comprising inducing the formation of epiblast cells from said inner cell mass cells.

33. The method of claim 27, wherein said pluripotent cells are embryonic stem cells.

34. The method of claim 33, further comprising inducing the formation of epiblast cells from said embryonic stem cells.

35. The method of claim 27, wherein said contacting of said pluripotent cells with said BMP proteins comprises co-culturing said pluripotent cells with cells selected from the group consisting of:
a) cells naturally secreting BMP4 and BMP8B;
b) cells naturally secreting BMP2 and BMP8B;
c) cells naturally secreting BMP 2, BMP4 and BMP8B;
d) a combination of cells naturally secreting BMP4 and cells naturally secreting BMP8B;
e) a combination of cells naturally secreting BMP2 and cells naturally secreting BMP8B;
f) a combination of cells naturally secreting BMP2, cells naturally secreting BMP4, and cells naturally secreting BMP8B;
g) transgenic cells secreting BMP4 and BMP8B;
h) transgenic cells secreting BMP2 and BMP8B;
i) transgenic cells secreting BMP2, BMP4 and BMP8B;
j) a combination of transgenic cells secreting BMP4 and transgenic cell secreting BMP8B;
k) a combination of transgenic cells secreting BMP2 and transgenic cells secreting BMP8B;
l) a combination of transgenic cells secreting BMP2, transgenic cells secreting BMP4, and transgenic cells secreting BMP8B; and
m) a combination of any of the above.

36. The method of claim 27, wherein said contacting of said pluripotent cells with said BMP proteins comprises addition of a primordial germ cell enhancing amount of BMP8B and BMP4, BMP2 or a combination of BMP2 and BMP2 to the medium containing said pluripotent cells.

37. The method of claim 27, wherein said contacting of said pluripotent cells with said BMP proteins comprises contacting said pluripotent cells with medium previously used to culture cells selected from the group consisting of:
a) cells naturally secreting BMP4 and BMP8B;
b) cells naturally secreting BMP2 and BMP8B;
c) cells naturally secreting BMP 2, BMP4 and BMP8B;
d) a combination of cells naturally secreting BMP4 and cells naturally secreting BMP8B;
e) a combination of cells naturally secreting BMP2 and cells naturally secreting BMP8B;
f) a combination of cells naturally secreting BMP2, cells naturally secreting BMP4, and cells naturally secreting BMP8B;
g) transgenic cells secreting BMP4 and BMP8B;
h) transgenic cells secreting BMP2 and BMP8B;
i) transgenic cells secreting BMP2, BMP4 and BMP8B;
j) a combination of transgenic cells secreting BMP4 and transgenic cells secreting BMP8B;
k) a combination of transgenic cells secreting BMP2 and transgenic cells secreting BMP8B;
l) a combination of transgenic cells secreting BMP2, transgenic cells secreting BMP4, and transgenic cells secreting BMP8B; and
m) a combination of any of the above.
